(19) United States
(12) Patent Application Publication
(10) Pub. No.: US 2005/0209220 A1
(43) Pub. Date: Sep. 22, 2005

(54) Treatment of peripheral neuropathy

(76) Inventor: Jeffrey Conforti, Franklin Lakes, NJ (US)

Correspondence Address: Ralph T. Lilore, 371 Franklin Avenue, PO Box 510, Nutley, NJ, 07110, US

(21) Appl. No.: 10/804,419

(22) Filed: Mar. 19, 2004

(51) Int. Cl. A61K 31/5415; A61K 31/335

(52) U.S. Cl. 514/225.8; 514/450

(57) ABSTRACT

The present invention centers around the treatment of peripheral neuropathy by administering to a symptomatic patient, especially one suffering pain and/or burning symptoms and especially in the legs or feet, and soles of the feet, a combination of two medications, a substituted phenothiazine, and a tricyclic antidepressant. The substituted phenothiazine potentiates the activity of, or acts synergistically with the tricyclic antidepressant, to provide relief that is otherwise not obtainable with one medication alone at reasonable dosage levels. The particular antidepressant may be imipramine (or analog thereof) and may be selected from the group consisting of the following well-known antidepressants: desipramine, imipramine, imipramine N-oxide, tripripramine, clomipramine, doxepin, amitriptyline, nortriptyline, protriptyline, and their pharmaceutically acceptable free forms, and acid addition salts and esters thereof. The second compound of the regimen is a substituted phenothiazine. Those preferred for use in the invention are selected from the group consisting of the following: chlorpromazine hydrochloride, mesoridazine besylate, thioridazine hydrochloride, acetophenazine maleate, fluphenazine, fluphenazine hydrochloride, fluphenazine enanthate, fluphenazine decanoate, perphenazine, trifluoperazine hydrochloride, and their pharmaceutically acceptable free forms, and acid addition salts and esters thereof. Most preferred is fluphenazine hydrochloride. The most preferred combination of antidepressant and substituted phenothiazine for use is desipramine hydrochloride with fluphenazine hydrochloride. The substituted phenothiazine may be taken alone, i.e. not in combination with the antidepressant. For fluphenazine, a dosage level higher than the amount used in the combination may be required depending on the severity of the neuropathy.

### CROSS REFERENCE TO RELATED APPLICATIONS

(Not Applicable).

### STATEMENT REGARDING FEDERALLY SPONSORED RESEARCH OR DEVELOPMENT

(Not Applicable).

### REFERENCE TO A SEQUENCE LISTING, A TABLE, OR A COMPUTER PROGRAM LISTING APPENDIX SUBMITTED ON COMPACT DISC (SEE 37 CFR 1.52(e)(5))

(Not Applicable).

### BACKGROUND OF THE INVENTION

Field of the Invention

This invention relates to the treatment of peripheral neuropathy and in particular to the relief of symptoms of peripheral neuropathy. More particularly, it relates to the treatment of peripheral neuropathy by the administration in combination of at least two different tricyclic organic compounds, one of which is a tricyclic compound such as an imipramine (or analog thereof) having a heterocyclic or homocyclic middle ring and the other of which is a substituted phenothiazine. The two different drugs are administered preferably orally and preferably at the same time, as will be discussed in more detail below. The invention also relates to a novel product comprising the two medicaments in a unit dosage package form.

Peripheral neuropathy is a term used to describe disorders of the peripheral nervous system. The peripheral nervous system includes nerves in the upper and lower extremities, e.g. toes, feet, legs, fingers, hands, arms, or face, torso, and some cranial nerves. In general, all of the nerves not located in the central nervous system (which includes the brain and the spinal cord) are considered to be peripheral nerves.

A number of factors can cause neuropathies. When a single nerve is affected, the most likely cause is trauma or some type of repetitive use that puts pressure on the nerve. Nerve pressure can result from using a cast or crutches, spending a long time in an unnatural position, such as typing at a computer keyboard, or having a tumor or abnormal bone growth.

When damage occurs to several nerves, the cause is frequently diabetes. About half of all people with diabetes develop some type of neuropathy. Other common causes include alcoholism, HIV/AIDS, inherited disorders, a deficiency of certain vitamins, especially the B vitamins, B-1 and B-12, and certain types of cancer, kidney, and liver diseases.

Autoimmune diseases, including lupus and rheumatoid arthritis, kidney disease, liver disease, and an underactive thyroid (hypothyroidism) also can damage peripheral nerves. So, too, can exposure to poisons, some toxic substances such as lead, mercury, arsenic, organic solvents, and carbon monoxide, and certain medications, especially those used to treat cancer or AIDS. One may even inherit a tendency to develop peripheral neuropathy.

Sometimes bacterial or viral infections may cause neuropathy. An acute condition called Guillain-Barre syndrome can cause severe damage to all or part of the peripheral nerves by destroying the myelin sheath that covers nerve fibers. The myelin sheath acts as an insulator for the nerves and helps conduct nerve impulses.

It is not always easy to pinpoint the cause of peripheral neuropathy. In fact, if one's neuropathy is not associated with diabetes, it is possible the cause may never be found.

Unfortunately, peripheral nerves are fragile and easily damaged. Damage to a peripheral nerve can interfere with the communication between the area it serves and the brain, affecting one's ability to move certain muscles or feel normal sensations. The symptoms will depend on the cause of the neuropathy and on which nerve or nerves are involved. If a sensory nerve is damaged, symptoms may include:
Pain, e.g. burning pain, sharp jabbing, electric-like pain, extreme sensitivity to touch
Numbness
Tingling
Loss of feeling
Unusual feeling, e.g. the sensation that one is wearing an invisible glove or sock
Lack of coordination

These symptoms often begin gradually and may start as a tingling sensation or numbness in the toes or the balls of the feet and spreads upward. Tingling might also begin in the hands and extend up the arms. In some cases, one's skin may become so sensitive that the slightest touch is agonizing. Severe cases can include a complete lack of feeling in the hands or feet.

At times, the symptoms may be barely noticeable, and some people go years without realizing anything is wrong. For others, symptoms are constant, and may be almost unbearable, especially at night.

Conventional treatment involves treating the underlying condition causing the neuropathy and repairing the damage, as well as treatment to provide symptom relief and management of pain.

Attempts at providing symptom relief often include the use of various medications, but in many cases, relief of symptoms, especially relief from pain or burning sensation, has not been easy to achieve.

The following medications have been used with varying degrees of success, but their use is not entirely satisfactory:
Pain relievers. OTC pain relievers, such as acetaminophen and nonsteroidal anti-inflammatory drugs (NSAIDs), such as aspirin and ibuprofen, usually help mild symptoms. For more severe symptoms, a doctor may recommend prescription NSAIDs. If taking NSAIDs for long periods of time or in large doses, one may develop nausea, stomach pain, bleeding, or even ulcers.
Anti-seizure medications. Drugs such as gabapentin, carbamazepine, and phenytoin, were originally developed to treat seizure disorders (epilepsy). However, doctors often also prescribe them for jabbing pain. Side effects may include drowsiness and confusion.
Lidocaine patch. This patch contains the topical anesthetic lidocaine. It is applied to the area where the pain is most severe, and can be used up to three patches a day to relieve pain.
Tricyclic antidepressants. Antidepressant medications, such as amitriptyline, nortriptyline, desipramine, and imipramine, may provide relief for mild to moderate symptoms by interfering with chemical processes in the brain that cause one to feel pain. Common side effects of these medications at antidepressant therapeutic dosages may include balance problems, dry mouth, nausea, tiredness or weakness, constipation, and weight gain.
Other medications. Opioid analgesics, such as codeine or oxycodone (OxyContin) may be used to relieve pain. However, this class of medications produces numerous side effects, including addiction, that make long-term use of these drugs undesirable. Mexiletine (Nexitil), a drug ordinarily used to treat irregular heart rhythms, sometimes helps relieve burning pain. The topical ointment capsaicin (Capzasin, Zostrix) may help ease the pain of diabetic neuropathy, and aside from a mild tingling, burning or slight skin irritation where the ointment is applied, it has few side effects.

The foregoing suffer from a variety of disadvantages, side effects, and unsatisfactory efficacy. The art would therefore be well-served by having available a regimen that provides more reliable symptom management, especially pain relief.

### DESCRIPTION OF THE RELATED ART INCLUDING INFORMATION DISCLOSED UNDER 37 CFR 1.97 AND 1.98

There is a discussion of peripheral neuropathy on the Mayo Clinic website www.mayoclinic.com. Much of the background information presented above is included at that website, including a statement that certain tricyclic antidepressants, i.e. amitriptyline, nortriptyline, and imipramine may provide relief for mild to moderate symptoms of peripheral neuropathy. We are unaware of any prior art which shows use of substituted phenothiazines either alone, or in combination with any tricyclic antidepressant, in the treatment of peripheral neuropathy.

### BRIEF SUMMARY OF THE INVENTION

The present invention centers around the treatment of peripheral neuropathy by administering to a symptomatic patient, especially one suffering pain and/or burning symptoms and especially in the legs or feet, and soles of the feet, a combination of two medications, a substituted phenothiazine, and a tricyclic antidepressant. The substituted phenothiazine potentiates the activity of, or acts synergistically with the tricyclic antidepressant, to provide relief that is otherwise not obtainable with one medication alone at reasonable dosage levels.

Specifically, as noted above, while certain tricyclic antidepressant drugs have been suggested for the treatment of pain in peripheral neuropathies (see the Mayo Clinic website), very often the relief from the symptoms is not obtained, or if obtained, is obtained at higher dosages at which side effects become more pronounced. This is the case with the antidepressant tricyclic compounds known as imipramines and “analogs” of imipramines. By “analog” is meant those compounds wherein the nitrogen heteroatom of the middle heterocyclic ring of imipramine is replaced with a carbon atom to form a homocyclic ring. These compounds are mainly used in the medical arts as potent antidepressants.

However, the use of these medicaments leaves much to be desired in achieving satisfactory outcomes in the relief of symptoms, especially the burning pain from peripheral neuropathy of the feet and legs. Often, to get effective relief, larger doses, potentially even approaching the antidepressant therapeutic dose, may be required with the onset of unpleasant side effects.

Nevertheless, the present invention utilizes the antidepressant compounds as one of the two medications used in the combination therapy. Thus, the invention is effective to increase or potentiate the activity of the antidepressant such that relief of pain and burning symptoms is obtained at lower dosages of the antidepressant than would be required if the antidepressant is used alone. This is achieved by administering with the antidepressant a second compound which is a member of a class of tranquilizers known as substituted phenothiazines. These compounds are well-known tranquilizers, but they have not, to our knowledge, been used to treat peripheral neuropathy.

Best results are obtained when the two medicaments are taken by the patient simultaneously, preferably orally. While oral administration is preferred, parenteral administration is also suitable. The term “simultaneous” is meant to apply to any administration which results in there being present in the body, at some point in time, therapeutically effective levels of both medications, even if they are not administered together. The best mode of doing this is by administering the drugs together. This is also the most convenient for the patient. However, “simultaneous” includes taking one drug after the other sufficiently close enough in time so that each drug is present in the body for a sufficient period of time to be effective. Thus, for example, if the tricyclic compound is administered first, the phenothiazine compound could be administered as much as an hour or more after the first tricyclic compound, the controlling concept being that the second compound be administered while the first compound is still present in the body in sufficient amounts to exert its therapeutic effect. The two drugs may also be blended together into one formulation in the appropriate dosages to provide a single formulation for administration.

In addition, the substituted phenothiazines may be used alone, without the imipramines using somewhat elevated dosages, depending on the severity of the symptoms.

The drugs used in combination in the invention are well-known in the art, are approved by the Food and Drug Administration (FDA) for their intended antidepressant and tranquilizing medication, and are available in the market in various forms, including pill forms containing various dosage levels, through prescription by physicians. The Physician's Desk Reference lists the available formulations and dosage levels.

### BRIEF DESCRIPTION OF THE SEVERAL VIEWS OF THE DRAWINGS

(Not Applicable).

### DETAILED DESCRIPTION OF THE INVENTION

The present invention provides a method for treating peripheral neuropathy and a novel unit dose package for use in such treatment. The method comprises the simultaneous administration to a person having peripheral neuropathy, of an effective amount of two compounds, an imipramine (or analog thereof) and a substituted phenothiazine, each either in free form or as a pharmaceutically acceptable acid addition salt, such as the hydrochloride, or as an ester thereof. By “effective amount” is meant an amount of the drugs which acting in combination in the presence of each other is effective to reduce the symptoms of the peripheral neuropathy to an acceptable level. The particular imipramine (or analog) may be selected from the group consisting of the following well-known antidepressant, non-proprietary name, compounds:
desipramine,
imipramine,
imipramine N-oxide,
tripripramine,
clomipramine,
doxepin,
amitriptyline,
nortriptyline,
protriptyline,

The second compound of the regimen is a substituted phenothiazine. Substituted phenothiazines are regarded as tranquilizers. Those preferred for use in the invention are selected from the group consisting of the following, non-proprietary name, compounds:
chlorpromazine hydrochloride,
mesoridazine besylate,
thioridazine hydrochloride,
acetophenazine maleate,
fluphenazine,
fluphenazine hydrochloride,
fluphenazine enanthate,
fluphenazine decanoate,
perphenazine,
trifluoperazine hydrochloride,
   and their pharmaceutically acceptable free forms, and acid addition salts and esters thereof. Most preferred is fluphenazine hydrochloride. The most preferred combination of antidepressant and substituted phenothiazine for use in this invention is desipramine hydrochloride with fluphenazine hydrochloride.

All of the foregoing named compounds are well-known in the pharmaceutical art and are marketed in unit dosage form for use in the medical field. The term “unit dosage form” used herein is a general term and is meant to apply to any form of the medicament such as a pill, tablet, capsule, powder, or the like, capable of being administered, preferably orally, to a patient and containing the appropriate amount of medicament for a given purpose, and intended to be used for a single dosage or as one of multiple dosages. The compounds are described herein by their art-known, non-proprietary generic names. Their chemical names, trade names, structures, therapeutic and pharmacologic information, and therapeutic category can be found in the literature such as, for example, in the Merck Index, 9^{th} Edition 1976, Goodman & Gilman, The Pharmacological Basis of Therapeutics, 9^{th} Edition 1996, and the Physician's Desk Reference 2004.

The substituted phenothiazines have, heretofore, not been known as effective for reducing the pain or burning of peripheral neuropathy, nor as having a potentiating effect on the tricyclic antidepressants allowing therapeutic symptom relief at lower dosages thereof in treating peripheral neuropathy. In this regard, it has been found that the use of the tricyclics alone at reasonable dosages has not been suitable in relieving the symptoms, especially pain or burning. When a substituted phenothiazine is added to the regimen as a therapeutic, it is possible to use low dosages of both ingredients to provide the relief from pain. The actual dosage for each component varies with the activity of the compound when each is used for its antidepressant or tranquilizer indication, as the case may be. It has been found that the ranges of the antidepressant employed in practicing the present invention correspond to the lower end (or a fraction thereof) of the therapeutic dosage for the drug in its usual antidepressant use. The usual antidepressant dosage for the tricyclic antidepressant component can be found in the literature. For example, Gilman and Goodman report that the ranges for the imipramines identified herein is generally 100-200 mgs/day. The analogs have ranges of 100-200 mg/day (amitriptyline), 75-150 mg/day (nortriptyline), 15-40 mg/day (protriptyline). In determining dosages for use in the invention, initially it is useful to select a dosage of 10-30% of the low end of the usual dosage of the antidepressant as an antidepressant. With regard to the amount of substituted phenothiazine used, it has been found that an amount corresponding approximately to the lower end of the tranquilizer effect range is suitable. Thus, for fluphenazine having a normal tranquilizing usage in the range of 2-30 mgs/oral dose/day, a potentiating amount for use herein is 1-2 mgs/day, when used in combination with an antidepressant, especially with desipramine. The presence of the substituted phenothiazine allows the dosage of the desipramine to be much lower, of the order of 10-20 mgs, than the usual therapeutic antidepressant dosage (100-200 mgs/day) to ease the pain of peripheral neuropathy in the legs or feet. For other phenothiazines, somewhat larger dosages may be used to be effective with the tricyclic compound. In the present invention, an effective amount of desipramine or the other imipramines for treating peripheral neuropathy is about 7-30 mgs/day, usually about 20 mgs/day (preferably divided into two doses of 10 mgs. each) and 0.5-4 mgs/day, preferably about 2 mgs/day (preferably divided into two doses of 1 mg each) of fluphenazine. Correspondingly higher dosages of the other phenothiazines may be employed.

Good results are usually obtained with total daily dose ranges of desipramine at 15-30 mgs/day together with fluphenazine at 0.5 to 4 mgs/day depending on the severity of the pain. At the high end of the ranges, it is often preferred to divide the daily dose into two equal portions taken at twelve-hour intervals. The most preferred regimen is 1 mg fluphenazine hydrochloride and 10 mgs desipramine hydrochloride, together every twelve hours. Appropriate adjustments may be made for other antidepressants and phenothiazines depending on how their usual therapeutic dosages relate to the imipramines and fluphenazines.

The preferred mode of treatment using the combination therapy is to direct the patient to take one tablet or pill of each of the tricyclic compound and the substituted phenothiazine compound (each having the appropriate dose for use in peripheral neuropathy) together every 12 hours, usually first at bedtime and then the next morning. If the dosage selected is insufficient, the regimen can be increased gradually to taking two or more tablets of each at the same twelve-hour intervals.

While taking the combination of tablets together is the best mode, the patient can take one or the other first followed by the remaining pill later. The best benefit, however, is obtained when the combination of drugs is present in the body together for the longest period of time and hence ingestion of the pills together (or a formulation containing both) or one followed quickly by the other is best. If any delay is desired, it should not be so long as to allow the therapeutic benefit to disappear or be significantly minimized before the other is taken.

The formulations containing the two drugs mixed together or packages comprising each of the drugs separately are novel products. Preferred for use is a sealed package, such as a blister pack, which comprises one pill of the tricyclic compound at an effective dosage level, preferably 7-30 mgs of active compound and one pill of the substituted phenothiazine comprising an effective dosage level, preferably 0.5 to 4 mgs of active compound. This constitutes a unit dosage package.

The most preferred package comprises a pill containing approximately 10 mgs of desipramine hydrochloride and a pill containing approximately 1-2 mgs of fluphenazine hydrochloride.

If fluphenazine is to be taken alone, i.e. not in combination with the imipramine, a dosage level higher than the 4 mgs/day may be required depending on the severity of the neuropathy. For mild to moderate symptoms, initial dosages from the higher end of the fluphenazine range such as about 4-15 mgs/day are usually selected, with increasing amounts given until a satisfactory effective dose is reached.

The following examples illustrate various embodiments of the invention.

### EXAMPLES

1. A male patient age 30 presented with severe neuropathy of the lower extremities secondary to HIV infection. The patient complained about severe burning pain in both legs and feet with especially severe pain at night preventing fitful sleep. The patient was instructed to take one tablet containing 10 mgs of desipramine and one tablet containing 1 mg of fluphenazine together orally twice per day, once at bedtime and once twelve hours later.

Upon follow-up two weeks later, the patient reported that the pains in his legs had virtually disappeared while he was taking the regimen prescribed and that he was able to sleep at night.

2. A female diabetic patient with severe pains in her legs reported that it was very difficult for her to sleep with the pain. At first, she was instructed to take the one-half the regimen described in Example 1, that is 10 mgs of desipramine and 1 mg of fluphenazine at bedtime.

Upon follow-up, she reported improvement in reduction of intensity of pain, but not complete elimination. She was therefore instructed to take an additional 10 mgs of desipramine and 1 mg of fluphenazine twelve hours after the bedtime administration.

Upon follow-up two weeks later, she reported essentially complete elimination of leg pain.

3. A male diabetic patient with neuropathy of the lower extremities was prescribed 10 mgs of desipramine alone orally every twelve hours. He did not take any substituted phenothiazine. He reported lack of effectiveness in reducing pain at the 20 mgs/day dosage level which persisted even at a higher dosage of 40 mgs per day.

4. A female diabetic patient with severe pain in the lower legs and feet was instructed to take 1 mg of fluphenazine hydrochloride orally every twelve hours beginning at bedtime. After two weeks, she reported somewhat of an improvement in pain reduction, but not complete elimination. When desipramine hydrochloride, 10 mgs/per twelve hour period, was taken with the 1 mg of fluphenazine, marked improvement was noted.

5. A male diabetic patient with severe lower extremity neuropathy manifested as pain in the lower legs and feet was instructed to take 2 mgs of fluphenazine hydrochloride orally every twelve hours beginning at bedtime. After two weeks, he reported somewhat of an improvement in pain reduction, but not complete elimination. He was instructed to double the daily regimen to 4 mgs every twelve hours and later reported substantial improvement in reducing the severity of pain.

1. The method for treating peripheral neuropathy which comprises simultaneously administering to a patient suffering from peripheral neuropathy an effective amount of an imipramine or an analog of imipramine selected from the group consisting of desipramine, imipramine, imipramine N-oxide, tripripramine, clomipramine, doxepin, amitriptyline, nortriptyline, and protriptyline, and pharmaceutically acceptable acid addition salts and esters thereof, and an effective amount of a substituted phenothiazine selected from the group consisting of chlorpromazine, mesoridazine, thioridazine, acetophenazine, fluphenazine, perphenazine, and trifluoperazine and pharmaceutically acceptable acid addition salts and esters thereof.

2. The method according to claim 1 wherein the route of administration is oral.

3. The method according to claim 2 wherein the substituted phenothiazine is fluphenazine.

4. The method according to claim 2 wherein the imipramine is desipramine.

5. The method according to claim 3 wherein the imipramine is desipramine.

6. The method according to claim 5 wherein the desipramine compound is present as the hydrochloric acid addition salt and the fluphenazine compound is present as the hydrochloric acid addition salt, the enanthate or the decanoate.

7. The method according to claim 6 wherein the amount of the desipramine compound administered is from 7 to 30 mgs/day and the amount of fluphenazine compound is from 0.5 to 4 mgs/day.

8. The method according to claim 7 wherein the desipramine compound and the fluphenazine compound are administered essentially at the same time.

9. The method according to claim 8 wherein the desipramine compound and the fluphenazine compound are in the form of separate unit dosage forms.

10. The method of claim 9 wherein the total dosage of each compound is divided into two daily dosages.

11. The method for treating peripheral neuropathy which comprises administering to a patient suffering from peripheral neuropathy an effective amount of a substituted phenothiazine selected from the group consisting of chlorpromazine, mesoridazine, thioridazine, acetophenazine, fluphenazine, perphenazine, trifluoperazine and pharmaceutically acceptable acid addition salts and esters thereof.

12. The method according to claim 11 wherein the route of administration is oral.

13. The method according to claim 12 wherein the substituted phenothiazine is fluphenazine, fluphenazine hydrochloride, fluphenazine enanthate, or fluphenazine decanoate.

14. The method according to claim 13 wherein the substituted phenothiazine is fluphenazine hydrochloride.

15. The method according to claim 14 wherein the amount of fluphenazine compound is from 0.5 to 4 mgs/day.

16. An article of manufacture comprising a first and a second compound for use in treating peripheral neuropathy wherein one of said compounds is an imipramine or an analog of imipramine selected from the group consisting of desipramine, imipramine, imipramine N-oxide, tripripramine, clomipramine, doxepin, amitriptyline, nortriptyline, and protriptyline, and pharmaceutically acceptable acid addition salts and esters thereof, and said second compound is a substituted phenothiazine selected from the group consisting of chlorpromazine, mesoridazine, thioridazine, acetophenazine, fluphenazine, perphenazine, trifluoperazine and pharmaceutically acceptable acid addition salts and esters thereof.

17. The article of claim 16 wherein said first compound and said second compound are each present as a discrete, orally administerable unit dosage form in a single package.

18. The article of claim 17 wherein the imipramine is desipramine.

19. The article of claim 18 wherein the substituted phenothiazine is fluphenazine.

20. The article of claim 19 wherein the desipramine is present as the hydrochloric acid addition salt and the fluphenazine is present as the hydrochloric acid addition salt, the enanthate or the decanoate form.

21. The article of claim 20 wherein each of the desipramine and the fluphenazine is present as the hydrochloric acid addition salt.

22. The article of claim 21 wherein the amount of the desipramine hydrochloride is from 7 to 30 mgs and the amount of fluphenazine hydrochloride is from 0.5 to 4 mgs.

23. The article of claim 22 wherein the amount of the desipramine hydrochloride is 10 mgs and the amount of fluphenazine hydrochloride is 1 mg.
